(19) United States
(12) Patent Application Publication
(10) Pub. No.: US 2012/0095515 A1
(43) Pub. Date: Apr. 19, 2012

(54) Cannulated Screw and Core Assembly

(76) Inventor: J. Adam Hamilton, Fergus Falls, MN (US)

(21) Appl. No.: 13/242,563

(22) Filed: Sep. 23, 2011

Related U.S. Application Data
(60) Provisional application No. 61/385,598, filed on Sep. 23, 2010.

(51) Int. Cl. A61B 17/86; A61B 17/88

(52) U.S. Cl. 606/304; 606/104

(57) ABSTRACT

A cannulated screw and core assembly includes a cannulated screw having an elongate shaft with a central bore including internal threads and a recess at a proximal end for receiving a tool for applying torque to the screw; and a complementary core shaped to be threaded into the central bore and having a frangible portion. Rotation of the core causes the core to separate at the frangible portion, resulting in a screw having a reinforcing core extending substantially along the internal bore of the screw. The disclosure includes a tool for attaching the disclosed cannulated locking screw to a bone fragment or plate. The tool may include a barrel shaped to receive the core assembly and includes a handle attached to a proximal portion of the barrel and a shank that extends through the barrel to engage an end of the core.

### BACKGROUND

This disclosure relates to surgical implants and, more particularly, to surgical screws that may be used to secure fractured bone fragments.

In the treatment of bone fractures, it is often necessary to secure the fractured bone fragments together with screws. In some instances, screws alone may be sufficient to connect the bone fragments. In other instances, it may be necessary to mount a metal plate on the surface of a bone fragment to provide support for the screw heads.

In order to insert the screws properly into the bone fragments to be joined, it may be necessary to employ a guide wire. A guide wire may be a relatively thin (on the order of 1.25 mm) stainless steel wire that is inserted along the axis that is to be followed by the screw. A guide wire may be inserted into and through bone fragments by a drill.

If guide wires are employed, cannulated screws may be used to join the bone fragments. A cannulated screw is a screw having a hollow central shaft. Both cortical and cancellous screws may be cannulated. Cannulated cancellous screws may be used for metaphyseal fractures, while cannulated cortical screws may be used as leg screws for fixation of diaphyseal fractures.

An advantage of using cannulated screws is that the hollow central shaft may be sized to permit the screw to be inserted over a guide wire or guide pin. This facilitates accurate placement of the cannulated screw in the bone fragments. Once the cannulated screw is properly sunk into the bone fragments, joining them together, the guide wire may be removed.

A disadvantage with cannulated screws is that the presence of a hollow central shaft reduces the cross-sectional area of metal of the screw along the shaft length, so that a cannulated screw may be weaker with respect to resisting stresses, such as bending stresses, than a comparable noncannulated or solid screw of the same shaft diameter.

Accordingly, there is a need for a cannulated screw that withstands bending and other stresses comparable to a noncannulated screw of the same diameter and material.

### SUMMARY

This disclosure is directed to a cannulated screw and core assembly that may provide superior resistance to bending and other stresses when compared to conventional cannulated screws of the same diameter and material. In one aspect, the cannulated screw and core assembly may include a cannulated screw having external threads and a hollow central shaft. The proximal end of the shaft may include an enlarged head having a hex-shaped recess for receiving a driving tool. The hollow center or bore of the shaft may include internal threads that may be located adjacent the hex recess at the proximal end.

The screw and core assembly may include a solid core having a central shaft having a frangible portion positioned along its length. In one aspect, the frangible portion may comprise a necked portion formed in the solid core. The core may include external threads located on a distal portion, which in one aspect may be adjacent the frangible portion. The proximal portion of the core may terminate in a hex head, and may include a threaded recess for receiving a driving tool.

In another aspect, the cannulated screw may be a cannulated locking screw having a shaft with threads substantially along its entire length, a hollow central bore, a flared head having external threads that may engage and lock into a plate and having a recess shaped to receive a star drive. Internal threads may be formed on the surface of the hollow central bore that may be adjacent the star drive recess. The complementary locking screw core may include a central shaft having a frangible portion and external threads adjacent the frangible portion and formed on a distal portion of the shaft. That core may terminate in a flared head at a proximal end having a star drive recess.

In both aspects of the disclosed assembly, once the cannulated screw has been threaded into place and the guide wire removed, the central core, which may be shaped to be inserted into the central bore, is inserted in the bore. The size and pitch of the external threads of the central core may be selected and positioned on the central core to register with and engage the internal threads of the hollow central bore. The central core is then rotated within the hollow central bore so that the external threads of the central core may engage and lock with the internal threads of the cannulated screw as the threads as the threads of the core “bottom out” or reach the end of the threaded section on the central bore of the screw.

Since at this point the central core can no longer be rotated or threaded into the central bore, further rotation of the central core may cause the core to break at the frangible portion, which is located so that the distal portion of the core may terminate at or below the hex head recess of the cannulated screw. The remainder of the core—the proximal portion—may then be removed from the hollow central shaft. The resulting assembly may be a cannulated screw having a central core retained in the central shaft of the screw by a threaded engagement and extending along the central bore so that the central core adds strength to the cannulated screw—comparable to a solid screw—and resistance to bending and other stresses. The hex recess of the cannulated screw is not obstructed by the core so that the entire assembly of cannulated screw and core may be backed out of the bone or plate if desired.

This disclosure also describes a tool that may be used for attaching a disclosed core to a disclosed cannulated screw. The tool may include a two-piece, elongated barrel having a central bore, and a screw core driving tool shaped to be inserted into the barrel. The barrel may include a handle to facilitate holding the barrel in a proper orientation during use, and to prevent overtorquing the cannulated screw during insertion of the core. The barrel may include an enlarged portion shaped to enclose the enlarged head of the screw core driving tool. The distal portion of the barrel may be shaped to be inserted into the hex-shaped recess of the proximal portion of a cannulated screw core.

The screw core driving tool includes an enlarged end that may include a hex-shaped recess shaped to receive the hex head of a core. The driving tool may include includes a shank shaped to rotate and translate longitudinally within the central bore.

Additional advantages of the disclosed screw and core assembly, and of the driving tool, may be apparent from the following description, the accompanying drawings and the appended claims.

### BRIEF DESCRIPTION OF THE DRAWINGS

FIG. 1 is a side elevation in section of one aspect of the disclosed cannulated screw of the cannulated screw and core assembly;

FIG. 2 is side elevation of a core of the disclosed cannulated screw and core assembly that is complementary to the cannulated screw of FIG. 1;

FIG. 3 is a side elevation in section of the cannulated screw of FIG. 1 with a distal portion of the screw core of FIG. 2 positioned within its hollow central bore;

FIG. 4 is a side elevation in partial section of another aspect of the disclosed cannulated screw and core assembly;

FIG. 5 is a side elevation of a core of a cannulated locking screw and core assembly, shaped to be received within the cannulated locking screw of FIG. 4;

FIG. 6 is a somewhat schematic, side elevation in partial section of a tool for attaching the disclosed screw core of the type shown in FIG. 2 to a cannulated screw of the type shown in FIGS. 1 and 3; and

FIG. 7 is a side elevation in section of the cannulated screw of FIG. 1 receiving the screw core of FIG. 2.

### DETAILED DESCRIPTION

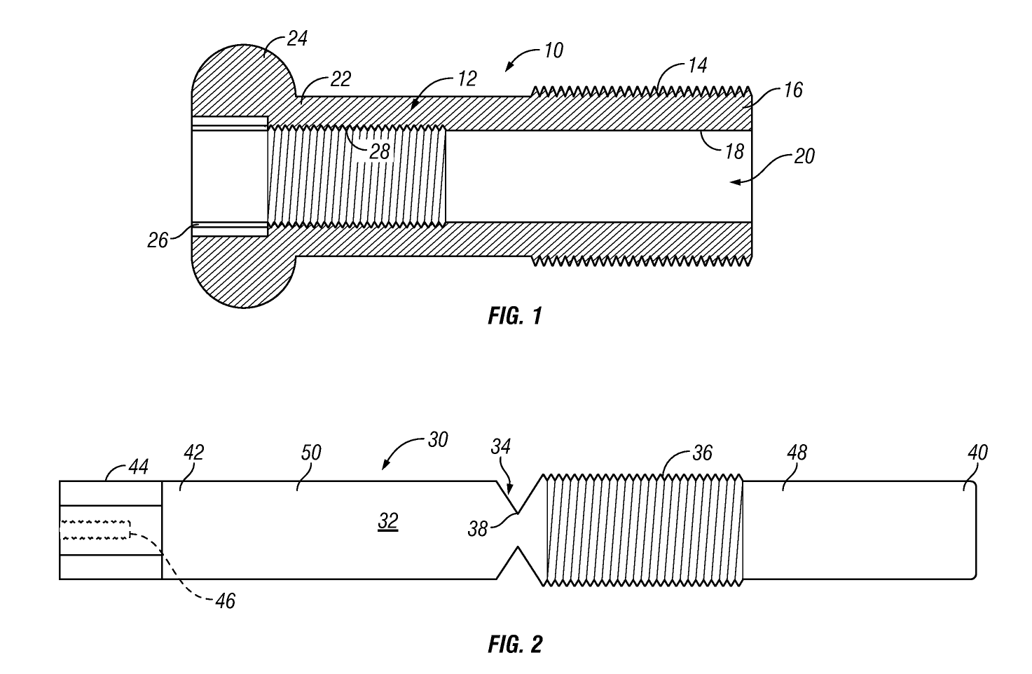

As shown in FIG. 1, one aspect of the disclosed cannulated screw system includes a cannulated screw, generally designated 10, having an elongated shaft 12 with external threads 14 at least about its distal portion 16. The screw 10 may include a substantially cylindrical inner surface 18 that defines a central bore 20. The central bore 20 may be shaped to receive a conventional guide wire (not shown) that, as discussed previously, is used to locate the cannulated screw 10 properly in bone to be repaired (not shown). The proximal portion 22 of the shaft 12 may include a head 24 having a recess 26 shaped to receive a hex-shaped driving tool (not shown), and which is formed about the bore 20. The head 24 may be enlarged in diameter relative to said shaft 12.

The cannulated screw 10 may be formed of stainless steel or other surgical steel alloys suitable for use in orthopedic procedures. In addition, the screw 10 may be formed of composites, or combinations of alloys and composites.

The cylindrical inner surface 18 of the screw 10 may include internal threads 28, which may be located adjacent the hex recess 26. The internal threads 28 are shown extending from a point adjacent the hex recess 26 toward the distal end 16 of the shank 12, but only part way to the distal end. However, in other embodiments the internal threads 28 may extend further toward the distal end 16, or extend the entire length of the inner surface 18.

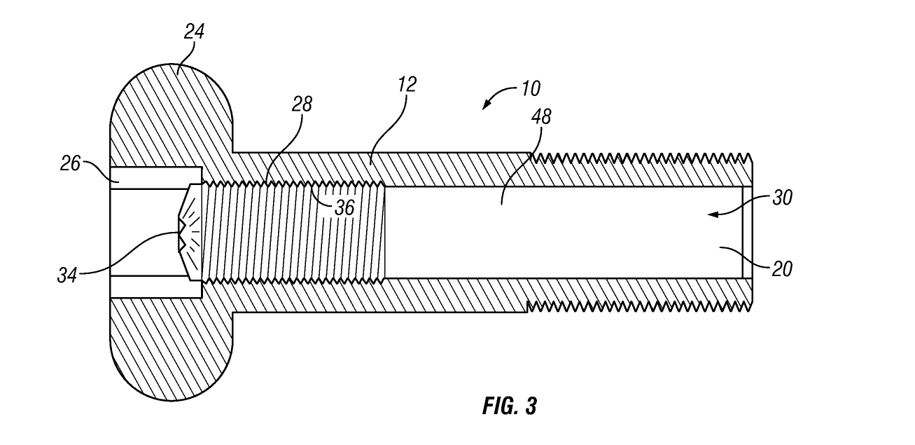

As shown in FIG. 2, a cannulated screw core, generally designated 30, includes a shaft 32 that may have a frangible portion 34 and external threads 36. The frangible portion 34 may comprise a reduced-diameter or necked portion 38 or other relatively weakened portion. The frangible portion 34 also may comprise a material different from that of the remainder of the core shaft 32 that is less resistant to fracture in response to torque than the remainder of the core shaft.

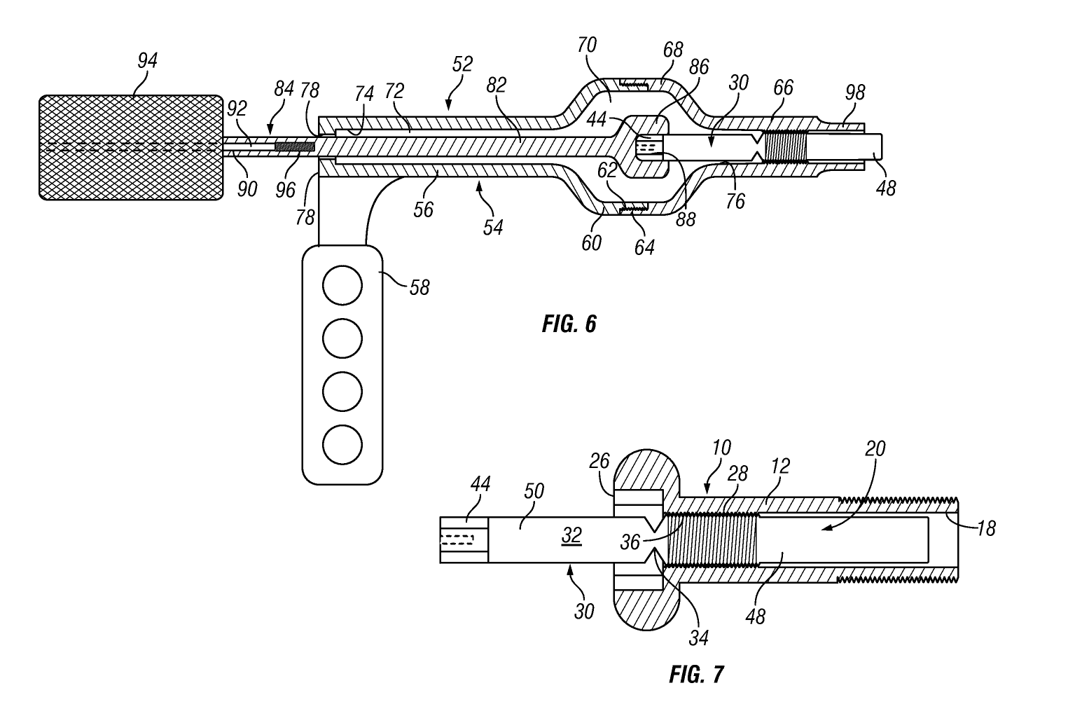

The external threads 36 may be positioned on the shaft 32 adjacent the frangible portion 34 and extending toward the distal end 40. The proximal portion 42 of the core may include a hex head 44 shaped to receive a driving tool (see FIG. 6, for example) and also may include a threaded recess 46, also shaped to receive a driving tool. The outer diameter of the shaft 32 may be sized and shaped so that the core may be inserted along the bore 20 of the cannulated screw 10 shown in FIG. 1.

Preferably, the frangible portion 34 is located at a point along the length of the core 32 to define a distal portion 48 of the core, which may extend from the frangible portion to the distal end 40, is shaped to be received within the shank 12 of the cannulated screw 10. As a result, the external threads 36 register with the internal threads 28.

As shown in FIG. 7, the screw core 30 is inserted into the bore 20 of the shank 12 of the cannulated screw 10 so that the external threads 36 of the distal portion 48 align and engage the internal threads 28 of the inner wall 18 of the screw. The external threads 36 may have a size and pitch to match the internal threads 28 of the bore 20. When this threaded engagement is achieved between the threads 28, 36, the proximal portion 50 of the core 30 protrudes rearwardly from the screw 10 and outwardly from the hex recess 26. The core 30 may be screwed into the bore 20 until the threads 36 reach the distal end of the threads 28. At that point the core bottoms out and further movement of the core 30 into the bore 20 is prevented. At this point the frangible portion 34 may be positioned adjacent the bottom of the hex recess 26. It is also within the scope of the disclosure to form the core shaft 32 of two discrete pieces, namely a proximal portion 42 and a distal portion 50. In such an embodiment, the frangible portion 34 may take the form of a relatively frangible connector, such as a frangible collar or an adhesive.

Further rotation of the core 30 causes the proximal portion 50 to separate from the distal portion 48 at the frangible portion 34, and the proximal portion 50 thereafter may be removed from the hex recess 26. As shown in FIG. 3, once the distal portion 50 (see FIG. 2) is removed, the frangible portion 34 is proximate the bottom of the hex recess 26 of the head 24 of the cannulated screw 10. The distal portion 48 of the core 30 extends substantially the entire length of the bore 20 and is retained therein by engagement of the external threads 36 with the internal threads 28 of the cannulated screw 10. Further, the hex recess 26 is not blocked, which allows removal of the screw 10 from the bone (not shown) or plate (not shown) in which it is mounted, if desired.

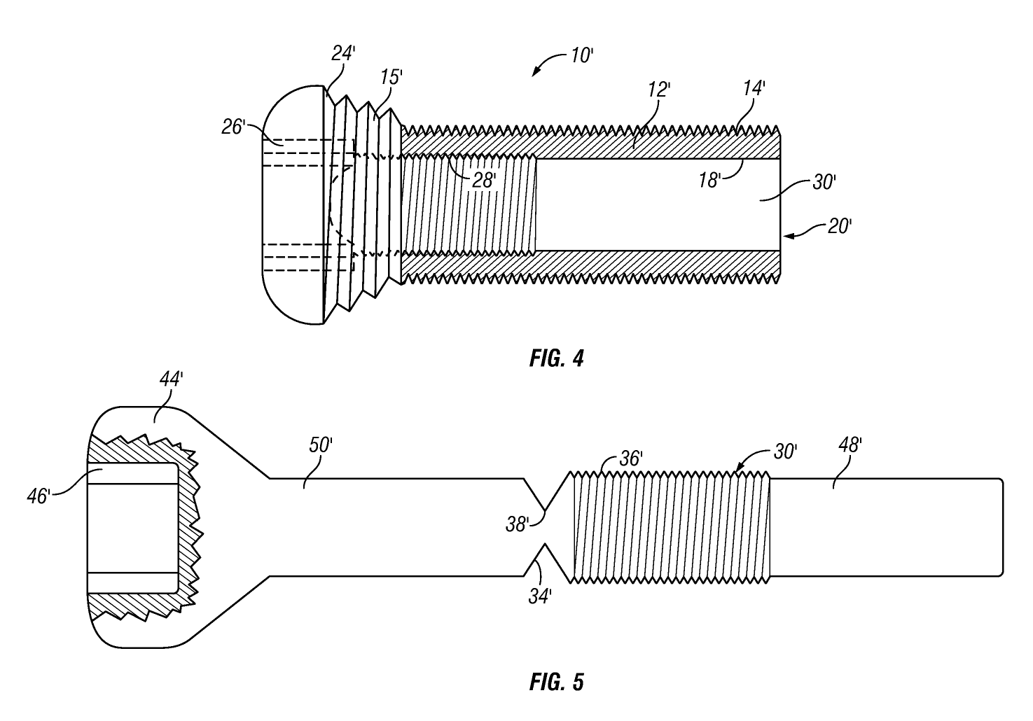

Another aspect of the disclosed cannulated screw is shown in FIG. 4 embodied in a cannulated locking screw 10′. Cannulated locking screw 10′ has a shaft 12′ that includes threads 14′ extending substantially the entire length of the shaft. A portion of the flared head 24′ includes threads 15′ for securing the cannulated locking screw to a metal plate (not shown) placed against the surface of a bone fragment that is to receive locking screw 10′. The shaft 12′ includes a cylindrical inner surface 18′ that extends substantially the entire length of the screw 10′ and defines a central bore 20′. The inner surface 18′ also includes interior threads 28′ adjacent a hex recess 26′ formed in the flared head 24′. With the embodiment of FIG. 4, the recess 26′ may communicate with the central bore 20′ and may be shaped to receive a star drive.

As shown in FIG. 5, the screw core, generally designated 30′, may include a distal portion 48′ having external threads 36′ that may be positioned adjacent to a frangible portion 34′. That frangible portion may include a necked portion 38′. The proximal portion 50′ may also include a flared head 44′ having a star drive recess 46′.

The engagement of the locking screw core 30′ within the bore 20′ of the cannulated locking screw 10′ of FIG. 4, shown in phantom in FIG. 4, is similar to that for the cannulated screw 10 and core 30 of FIGS. 1 and 2, shown in FIG. 7. However, it should be noted that with the locking screw 10′, engagement of the cannulated locking screw with an associated plate provides a countertorque sufficient to prevent overtorquing or overtightening of the locking screw in associated bone fragments that might otherwise result from torquing the locking screw core 30′ into the cannulated locking screw.

As shown in FIG. 6, a tool, generally designated 52, may be employed to attach the core 30 to the cannulated screw 10, and remove the proximal portion 50 of the core, while preventing overtorquing of the screw in associated bone fragments. The tool 52 may include a tubular barrel 54 having a proximal portion 56 that is attached to and supported by a hand grip 58. The proximal portion 56 may have an enlarged end 60 with a threaded tip 62 that receives the corresponding threaded tip 64 of a distal portion 66. The distal portion 66 also may include a bell end 68 that carries the threaded tip 64.

The engagement of the bell end 60 of the proximal portion 56 with the bell end 68 of the distal portion 66 may create an enlarged, enclosed chamber 70 within the bore 72 of the barrel 56. The bore 72 is defined by the interior walls 74 and 76 of the proximal and distal portions 56, 66, respectively. The proximal portion 74 of the bore 72 may include an opening 78 formed in the base 80 of the barrel and shaped to receive the shank 82 of a driver, generally designated 84. The shank 82 terminates in an enlarged hex driver head 86 that may include a recess 88 shaped to receive the hex head 44 (see FIG. 2) of a core 30. The chamber 70 is sized to allow the hex driver head 86 to displace the core 30 longitudinally along the distal portion 66 until the distal portion 48 of the core 30 to be inserted into a cannulated screw 10 to the extent shown in FIG. 7. This may require the chamber 70 to be elongated longitudinally more than is shown in FIG. 6. In the alternative, the tool 52 may be used with a core that is sufficiently long to protrude from the end of the barrel (e.g., the entire distil portion of the core) to be inserted into a bore of a cannulated screw, so that only a short travel is required to insert the core into the bore.

The shank 82 also may include a recess 90 shaped to receive the shaft 92 of a detachable, knurled handle 94. The handle shaft 92 may be retained within the recess 90 by a threaded engagement 96. The distal portion 66 of the barrel 54 may include a hex-shaped tip 98 sized to be inserted into the hex recess 26 of a cannulated screw (see FIG. 1).

The deployment of the cannulated screw 10 and complementary core 30 assembly in the bone or bone fragment of a human or other mammal is as follows. Once the cannulated screw 10 has been mounted to a bone or bone fragment, or mounted through a metal plate, the guide wire (not shown), over which the cannulated screw may be slid, may be removed. The core 30 may be inserted into the shaft 72 of the barrel 56 so that hex head 44 of the core is received within the hex recess 88 of the tool 84. A portion of the distal portion 48 of the core protrudes from the tip 98 of the tool 52.

The tool may then be attached to the hex recess 26 of the head 24 of the screw 10 by inserting the hex tip 98 into the hex recess 26. Once the tool 52 has been attached in that manner, the handle 58 may be grasped by a user with the left hand to maintain a proper torque with respect to the cannulated screw 10. The user may grasp the handle 94 with the right hand and push the shank 82 in a distal direction to insert the core 30 into the bore 20 of the screw 10. Once the external threads 36 of the core begin to engage the internal threads 28 of the screw, the user rotates the handle 94 in a clockwise direction to create a threaded engagement between the core 30 and the screw 10.

To prevent overtightening of the screw 10 within the bone, in one embodiment the threads 62, 64 that interconnect the distal portion 66 of the barrel 54 with the proximal portion 56 are left-handed threads, so that the user may, if necessary, rotate the tool slightly counterclockwise, or at least apply a counterclockwise force on the tool. Because the tool 52 engages the screw at hex tip 98, this counterclockwise force will prevent the clockwise rotation of the core 30, which will transmit a clockwise rotating torque from the threads 36 of the core engaging the threads 28 of the screw 10, once the core threads bottom out on the screw threads, from overtightening the screw, which might strip the screw within the bone. Overtightening also might occur as a result of the friction between the engagement of the threads of the core and screw.

Once the threads 36 of the core 30 bottom out with respect to the internal threads 28 of the core, additional torque causes the core to break at the frangible portion 34, thus separating the proximal portion 50 from the distal portion 48. At this point, the user may withdraw the shank 82 relative to barrel 54 to separate the proximal portion 50 from the distal portion 48 further. The user then removes the tool 52 from engagement with the hex recess 26 of the screw 10, with the proximal portion 50 still engaging the hex recess 88 of the driver head 86. The resultant structure is as shown in FIG. 3.

The user may then displace the shank 82 forwardly within the tool to expose the proximal portion 50 so that it may be removed from engagement with the hex recess 88. In the alternative, the distal portion 66 of the barrel 54 may be removed from engagement with the proximal portion 56, and the proximal portion 50 of the core 30 removed from the hex recess 88, which would be exposed by the removal of the distal portion 66 therefrom.

Other forms of screw and core may be employed and not depart from the scope of the disclosure. For example, means other than an engagement between internal threads 28 and external threads 36 may be utilized to prevent rotation of the core relative to the screw, whereby rotation of the core relative to the screw effects fracture at the frangible portion and separation of the proximal portion of the core from the distil portion. Such other means may comprise longitudinal splines formed on the core that may engage or be received within longitudinal slots formed in the inner surface of the bore. Either structure may be considered a locking device that prevents relative rotation between the core and screw.

What is claimed is:

1. A cannulated screw assembly comprising:
a cannulated screw including an elongate shaft with a bore having internal threads; and
a core shaped to fit within said bore and having external threads shaped to engage said internal threads, and a frangible portion separating said core into a distil portion and a proximal portion, said core having a length sufficient to extend along said bore and reinforce said elongate shaft.

2. The cannulated screw assembly of claim 1 wherein said core has a length such that at least a portion of said proximal portion protrudes from said bore when said core is inserted into said bore.

3. The cannulated screw assembly of claim 1 wherein said screw includes a head having a recess shaped to receive a driving tool.

4. The cannulated screw assembly of claim 3 wherein said recess is shaped to receive a hex-shaped driving tool.

5. The cannulated screw assembly of claim 3 wherein said head is enlarged in diameter relative to said shaft.

6. The cannulated screw assembly of claim 4 wherein said core is shaped such that said frangible portion is positioned within said recess when said core is inserted within said bore.

7. The cannulated screw assembly of claim 6 wherein said external threads are positioned along said core on said distil portion.

8. The cannulated screw assembly of claim 7 wherein said external threads are positioned adjacent said frangible portion.

9. The screw assembly of claim 8 wherein said external threads extend along less than an entire length of said core.

10. The cannulated screw assembly of claim 9 wherein said external threads extend along less than an entire length of said distil portion.

11. The cannulated screw assembly of claim 1 wherein said distil portion includes an end shaped to receive a driving tool.

12. The cannulated screw assembly of claim 1 wherein said frangible portion includes a portion of reduced diameter relative to a remainder of said core.

13. The cannulated screw assembly of claim 1 wherein said frangible portion includes a portion less resistant to torque than a remainder of said core.

14. A cannulated screw assembly comprising:
a screw including an elongate shaft with a bore having a first locking component; and
a core shaped to fit within said bore and having a second locking component shaped to engage said first locking component, and a portion for separating said core into a distil portion and a proximal portion, said core having a length sufficient to extend along said bore and reinforce said elongate shaft.

15. The cannulated screw assembly of claim 14 wherein said first and second locking components are shaped to prevent relative rotation between said screw and said core.

16. The cannulated screw assembly of claim 14 wherein said portion for separating includes a portion of reduced diameter on said core.

17. The cannulated screw assembly of claim 14 wherein said portion for separating includes a frangible portion.

18. A method of using a cannulated screw assembly comprising:
providing a screw including an elongate shaft with a bore having a first locking component;
providing a core shaped to fit within said bore and having a second locking component shaped to engage said first locking component, and a portion for separating said core into a distil portion and a proximal portion, said core having a length sufficient to extend along said bore and reinforce said elongate shaft;
screwing said screw into a bone of a mammal;
inserting said core into said bore such that said first and second locking components engage;
rotating said core relative to said screw until said proximal portion separates from said distal portion; and
removing said proximal portion from said screw, such that said distil portion remains in said core.

19. A tool for attaching a cannulated screw assembly of claim 12 to a bone or bone fragment of a mammal comprising:
a hollow barrel shaped to receive a core therein at an end thereof; and
a rod having a first end shaped to be grasped by a user thereof, and a second end shaped to engage a proximal portion of said core, said rod being slidably and rotatably positioned within said hollow barrel.

20. The tool of claim 19 wherein said hollow barrel includes an end shaped to engage a recess in a head of a cannulated screw, such that rotation of said barrel causes said engaged cannulated screw to rotate.

21. The tool of claim 19 wherein said barrel includes a handle.
